(19) United States
(12) Patent Application Publication
(10) Pub. No.: US 2004/0053950 A1
(43) Pub. Date: Mar. 18, 2004

(54) Antimalarial activities and therapeutic properties of febrifugine analogues

(76) Inventors: Suping Jiang, Potomac, MD (US); Thomas Hudson, Bethesda, MD (US); Wilbur Milhous, Germantown, MD (US)

Correspondence Address: Elizabeth Arwine, Esq., USAMRMC, 504 Scott Street, Fort Detrick, MD, 21702, US

(21) Appl. No.: 10/600,446

(22) Filed: Jun. 20, 2003

Related U.S. Application Data
(60) Provisional application No. 60/390,334, filed on Jun. 20, 2002.

(51) Int. Cl. A61K 31/517

(52) U.S. Cl. 514/266.22

(57) ABSTRACT

Malaria is the most severe tropical parasitic disease that has caused millions of deaths in many countries. The threat of growing drug-resistant parasites requires development of new antimalarial drugs to overcome the emergence of resistance and to control the disease. Febrifugine is the active principle extracted from the Chinese herb Chang Shan (Dichroa febrifuga Lour) that has been used to treat malaria for more than two thousand years. Studies on the efficacy have been hindered due to the emetic effects of febrifugine. The present invention discloses febrifugine, halofuginone and febrifugine derivatives for use as antimalarial agents without the severe emetic effects observed in direct herbal use.

This application claims priority benefit from Provisional Application No. 60/390,334, filed Jun. 20, 2002.

### STATEMENT OF GOVERNMENT INTEREST

The invention described herein may be manufactured, used and/or licensed by or for the United States Government.

### BACKGROUND OF THE INVENTION

Malaria, one of the most endemic of infectious diseases exists in over one hundred countries, with concentrations in the tropical areas of Africa, Asia and Latin America. The World Health Organization reports that malaria is responsible for over one million deaths and disables over 42 million people worldwide. See World Health Report 2002, www.who.org.

The World Health Organization also reports that the incidence of malaria epidemics is increasing due to non-immune persons coming in contact with asymptomatic carries of the disease from endemic regions of the world. See id. This has particular impact on U.S. armed personnel and civilians situated in these tropical areas as they often lack a natural immunity against infection.

Those likely to be exposed to malaria are usually given prophylactic drugs such as chloroquine, mefloquine, doxycycline or sulphadoxine-pyrimethamine. These drugs cannot guarantee full protection and often act to lessen the severity of the symptoms in infected persons. More importantly, newer and deadlier parasites are resistant to these treatments. The urgent need for the treatment of malaria is largely dependent on the discovery and development of new antimalarial drugs.

Many scientists worldwide are striving to search for new antimalarial drug leads using different state-of-the-art approaches such as technologies of combinatorial chemistry, DNA microarray and high throughput screen. While these approaches may lead to antimalarial drugs, significant discoveries are still pending.

Ancient Chinese pharmacopoeia discuss the medical use of an ancient Chinese herb Chan Shan (Dichroa febrifuga Lour) for treatment against fevers caused by malaria, stomach cancer, expectorant, emetic and febrifuge with side effects of nausea and vomiting. See Li; Pen Tsao Kang Mu; 1596. Febrifugine{(2′S,3′R)-3-[3-(3-hydroxy-2-piperidyl)acetonyl]-4(3H)quinazolinone}, isolated from this plant over 50 years ago, was later identified as a quinazoline derivative having a molecular structure of C₁₆H₂₁O₃N₃. See Kuehl, et al.; Alkaloids of Dichroa febrifuga Lour; J. Am. Chem Soc., 70, 2091-2093; 1948. 2 The purified febrifugine displayed very potent antimalarial activity, 100 times as active as quinine against Plasmodium lophurae in duck models. But severe gastrointestinal injury was also observed in chicken models when over-lethal dosages were administered. See Jang, et al.; Chang Shan, A Chinese Antimalarial herb; Science, 103:59, 1946.

Notorious emetic activity exhibited by Dichroa febrifuga Lour and the gastrointestinal lesions caused by febrifugines in chicken models have for decades hampered further investigation of febrifugines in the mechanisms of action, clinical effectiveness and safety. Since the late sixties, however, natural febrifugines have been used as lead structures for the synthesis of many analogues in an attempt to reduce toxicity without compromising antimalarial activity.

Takaya, et al., recently found that chemical modification of febrifugine decreased the toxicity without hampering antimalarial affects. See Takaya, et al.; New type of febrifugine analogues, bearing a quinolizidine moiety, show potent antimalarial activity against Plasmodium malaria parasite; J. Med Chem. 42:3163-6; 1999. No toxic evidence was observed on the change of body and liver weight as well as hepatic marker enzymes when antiparasitic dosages were administered to infected mice for ten days. See Murata, et al.; Potentiation by febrifugine of host defense in mice against Plasmodium berghei NK65.; Biochem Pharmacol.; 58: 1593-601; 1999. He also found that febrifugine altered the production of nitric oxide and tumor necrosis factor-a in mouse macrophages. See Murata et al.; Enhancement of NO production in activated macrophages in vivo by an antimalarial crude drug, Dichroa febrifuga.; J. Nat. Prod. 61: 729-33; 1998. These studies indicate that febrifugines are not only promising antimalarial leads without apparent toxic effect as previously believed, but also possess unique antimalarial mechanisms that require further study.

Halofuginone {7-bromo-6-chloro-3-[3-hydroxy-2-piperidyl)-2-oxopropyl]-4(3H)-quinazolinone}, synthesized in the late 1960s as a potential antimalarial agent, is one of the febrifugine analogues with a chloride and bromide added at the position 6 and 7 on the quinazoline moiety. See Ryley, et al.; Chemotherapy of chicken coccidiosis; In Advances in Pharmacology and Chemotherapy; 10: 221-93; 1973. Halofuginone hydrobromide is a an FDA-approved feed additive commercially known as Stenorol, that has been widely used in the poultry industry to prevent coccidiosis in broiler chickens and growing turkeys for nearly twenty years. An incidence of overdose of Stenorol led to the discovery that halofuginone blocked the synthesis of cellular collagen by inhibiting collagen type I gene expression. See Halevy, et al.; Inhibition of collagen type I synthesis by skin fibroblasts of graft versus host disease and schleroderma patients: effect of halofuginone; Biochem Pharmocol; 52: 1057-63; 1996.

The inhibition of collagen type I gene expression has lead to intensive preclinical studies and rapid drug development for the control of many diseases relevant to the excessive synthesis of collagen. For example, halofuginone has been shown in animal models to reduce pulmonary fibrosis, prevent liver cirrhosis, reduce peritendinous fibrous adhesions after surgery, accelerate wound repair and prevent injury-induced arterial intimal hyperplasia. See Nagler, et al.; Reduction in pulmonary fibrosis in vivo by halofuginone; Am. J. Respir Crit Care Med.; 154:1082-6; 1996; Pines, et al.; Halofuginone, a specific inhibitor of collagen type I synthesis, prevents dimethylnitrosamine-induced liver cirrhosis; J. Hepatol.; 27: 391-8; 1997, Nyska, et al. Topically applied halofuginone, an inhibitor of collagen type I transcription, reduces peritendinous fibrous adhesions following surgery; Connect Tissue Res. 34: 97-103; 1996, Abramovitch, et al., Inhibition of neovascularization and tumor growth, and facilitation of wound repair, by halofuginone, an inhibitor of collagen type I synthesis; Neoplasia; 1:321-9; 1999 and Liu et al.; Halofuginone inhibits neointimal formation of cultured rat aorta in a concentration-dependent fashion in vitro; Heart Vessels; 13: 18-23; 1998. Moreover halofuginone has been shown to inhibit vascular tube formation out of rat aortic rings and the growth of mouse bladder carcinoma cells due to the interruption of collagen synthesis that provides an attractive new target for cancer therapy, especially when both activities-antiangiogenic and antimetastic are combined in the same module. See Elkin, et al.; Inhibition of bladder carcinoma angionenesis, stromal support, and tumor growth by halofuginone; Cancer Res.; 59:4111-8; 1999; and Elkin, et al.; Inhibition of matrix metalloproteinase-2 expression and bladder carcinoma metastasis by halofuginone; Clin. Cancer Res.; 5: 1982-8; 1999. It has also been reported that topical treatment of a chronic graft-versus-host disease patient with halofuginone caused an attenuation of skin collagen accompanied by increased neck rotation on the treated side. See Nagler, et al.; Topical treatment of cutaneous chronic graft versus host disease with halofuginone: a novel inhibitor of collagen type I synthesis; Transplantation; 68:1806-9; 1999. Based on these results, halofuginone is being tested against scleroderma in clinical trials in the United Kingdom and for a rapid development program against cancer at the National Institutes of Health. See Pines, et al.; Halofuginone: from veterinary use to human therapy; Drug Development Research; 50: 371-378; 2000.

U.S. Pat. Nos. 6,420,371B1 and 6,028,075 to Pines, et al., and U.S. Pat. No. 6,090,814 to Nagler, et al., incorporated herein by reference, disclose and claim halofuginone for reducing the progression of tumor formation and the inhibition of angiogenesis. While these references further substantiate the efficacy of halofuginone in treatment of various diseases, they are not directed to the treatment and use as antimalarial agents.

The promising results in the therapies discussed above have led a new and novel application of halofuginone and febrifugine derivatives discussed below.

### SUMMARY OF THE INVENTION

It is therefore, an objective of the invention to use quinazolinone compounds that are effective for the treatment of parasitic infections, specifically protozoan infections of the genus Plasmodium.

It is yet another objective of the invention to use halofuginone for the treatment against Plasmodium falciparum and Plasmodium berghei.

It is also another objective of the invention to use febrifugine/isofebrifugine extract obtained from the Chang Shan (Dichroa febrifuga Lour) plant for the treatment against Plasmodium falciparum and Plasmodium berghei.

It is yet another objective of the invention to use febrifugine derivatives as effective treatment against Plasmodium falciparum and Plasmodium berghei.

It is yet another objective to administer the quinazolinone compounds in a pharmaceutically effective amount orally, subcutaneously, intramuscularly, or intraperitonealy.

These and other objectives are discussed herein below.

### DESCRIPTION OF THE DRAWINGS

FIG. 1(a) shows Chang Shan (Dichroa febrifuga Lour) in plant form

FIG. 1(b) shows Chang Shan (Dichroa febrifuga Lour) in root form.

FIG. 1(c) shows NMR confirmation of Chang Shan root extracted febrifugines.

FIG. 1(d) shows TLC of purified febrifugine reaction.

FIG. 1(e) shows TLC of purified febrifugine to isofebrifugine.

FIG. 2(a) shows the in vitro drug susceptibility assay results.

FIG. 2(b) shows the in vitro drug toxicity assay results.

FIG. 3(a) shows a bar graph of febrifugine oral adminstration data.

FIG. 3(b) shows a bar graph of febrifugine subcutaneous administration data.

FIG. 3(c) shows survival data of mice subjected to febrifugine oral administration.

FIG. 3(d) shows survival data of mice subjected to febrifugine subcutaneous administration.

FIG. 4(a) shows necropsy analysis of death caused by malarial parasites versus drug toxic effects.

FIG. 4(b) shows effects of toxic doses of febrifugine.

FIG. 4(c) shows a comparison of toxic effects between oral and subcutaneous administration.

FIG. 5(a) shows the survival duration of Halofuginone treated mice.

FIG. 5(b) shows parasitemia on Day 6 in halofuginone treated mice.

### DETAILED DESCRIPTION OF THE INVENTION

Protozoan parasites of the genus Plasmodium are responsible for Malaria. The disease is transmitted to humans and animals through parasite-infected blood-feeding female mosquitoes. As the infected mosquitoes ingest bloodmeal from a human or animal host, they transmit the parasites to the human host, where it grows in the liver and then in infected red blood cells. Mosquitoes ingest the infected bloodmeal, provide another reproductive cycle for the parasite and then transmit it to other humans and animals.

The present invention is directed to the use of febrifugine, halofuginone and febrifugine/halofuginone derivatives for better control and treatment of parasitic protozoan infections, specifically, malarial parasites and diseases. Studies indicate that halofuginone and febrifugine analogs show inhibition to malarial recombinant kinases (PfMRK and PfMP-2) indicating interference to the parasite cellular signal transduction systems. The structures of febrifugine, halofuginone and the derivatives are given below.

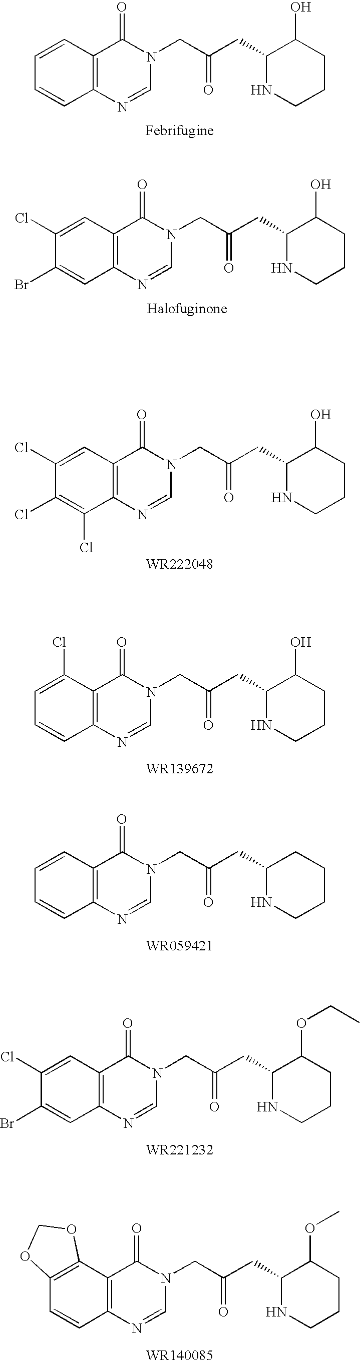

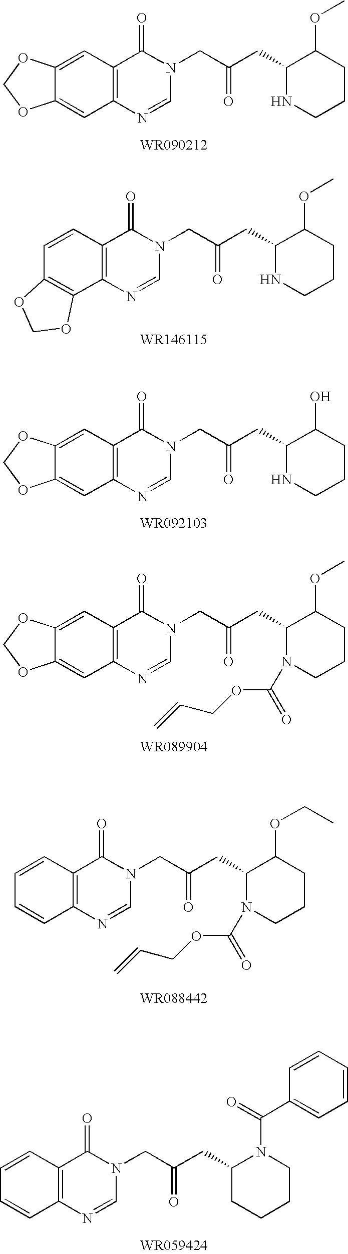

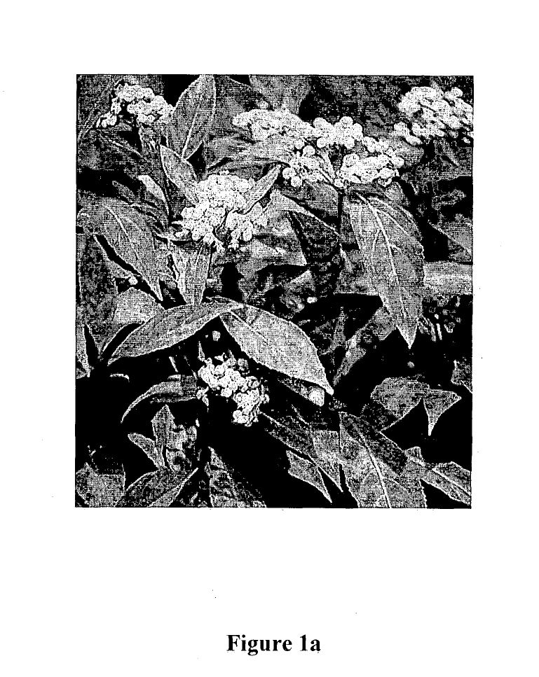

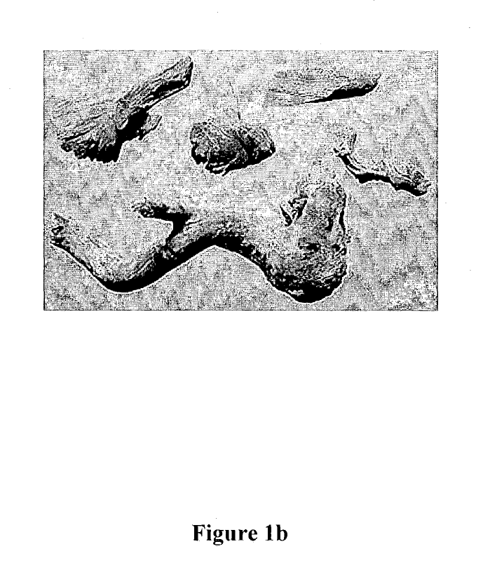

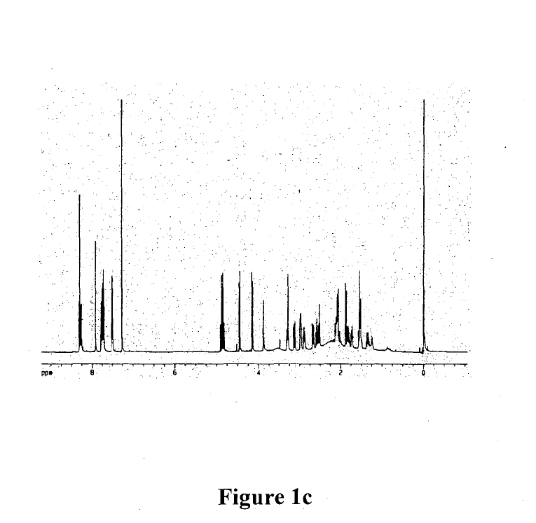

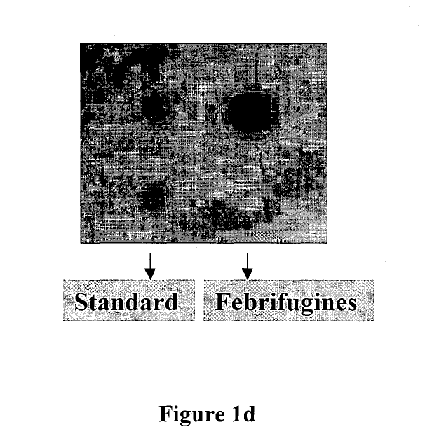

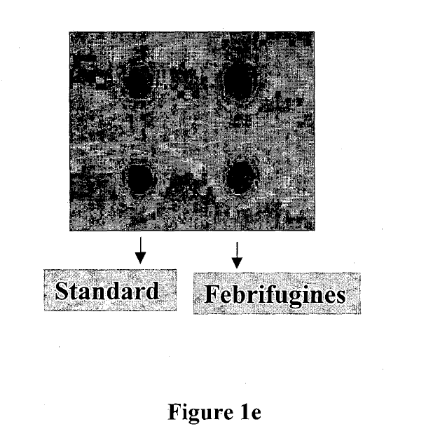

Febrifugine is the active principle extracted from the roots of the Chinese herb Chang Shan (Dichroa febrifuge Lour). The applicants of the present invention isolated febrifugine from Chang Shan, shown in plant and root form in FIGS. 1(a) and 1(b) respectively, to confirm that the herb did contain febrifugine. The compound was extracted from Chang Shan roots via methanol extraction mixed with 0.1 M HCl. Thereafter, the extract was chloroform partitioned. The pH of the aqueous layer was then adjusted to 9.5 with NH₄OH. The alkaloids where then extracted with CHCl₃ from the aqueous part. Thereafter the alkaloids were passed through a silica column with a petroleum Ether/Ethyl Acetate Wash. The febrifugines were then eluted by CHCl₃/MeOH. As shown in FIG. 1(c), NMR analysis confirmed that the extracted compounds were febrifugines with an above 95% purity. TLC analysis, as shown in FIGS. 1(d) and 1(e), shows that the purified febrifugines isomerized into 1:1 ratio of febrifugine and isofebrifugine when kept in methanol at room temperature overnight. The chemical structures of febrifugine and its isomer is as shown below:

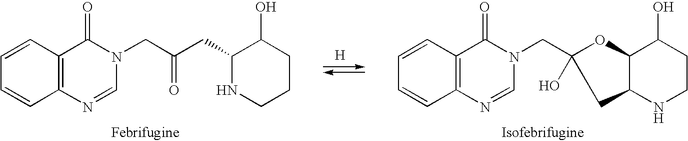

Febrifugine displayed very potent in vitro antimalarial activity, ten times stronger than chloroquine and artemisinin. Eight of the analogues, as shown in table 1 were also found to be very active against malarial parasites in culture.

TABLE 1
The antimalarial activities of febrifugine analogues
against P. falciparum (IC₅₀:ng/ml):

| Analogues | W2 strain | D6 strain |
|---|---|---|
| WR222048 | 1.292 | 0.962 |
| WR139672 | 1.626 | 1.228 |
| WR059421 | 26.014 | 17.111 |
| WR221232 | 17.196 | 11.109 |
| WR088442 | 287.577 | 202.887 |
| WR140085 | 14.05 | 10.468 |
| WR089904 | 129.403 | 89.727 |
| WR090212 | 25.63 | 18.105 |

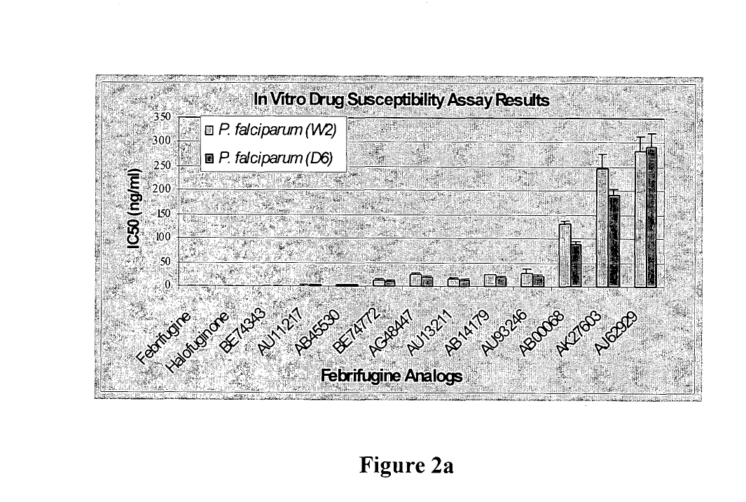

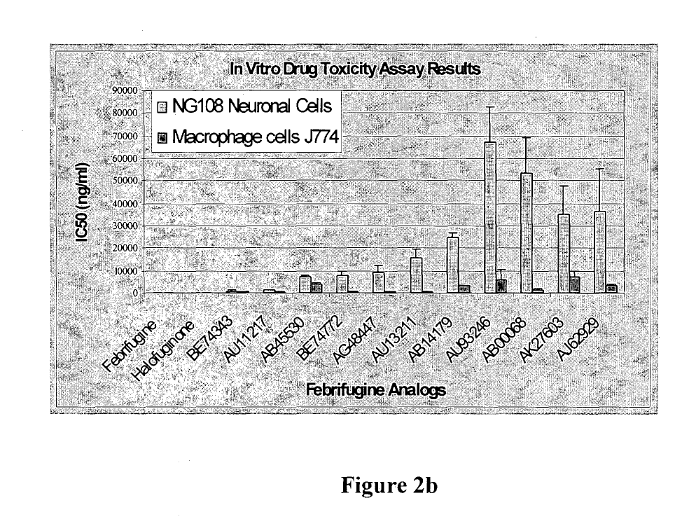

Febrifugines and their analogues also had lower toxicity to mammalian neuronal and macrophage cells. As shown in FIGS. 2(a) and 2(b), mammalian neuronal cells appear to notably be less susceptible to febrifugine analogues. Table 2 below summarizes the susceptibility results below.

TABLE 2
Susceptibility of mammalian cell lines to the analogues of
Febrifugine (IC₅₀:ng/ml).

| | Macrophage cells | Neuronal cells |
|---|---|---|
| Analogues | (J774) | (NG108) |
| WR222048 | 110 | 1098.5 |
| WR139672 | 69 | 1314 |
| WR059421 | 236 | 5380.5 |
| WR221232 | 286.5 | 5990.5 |
| WR088442 | 9097 | 21500 |
| WR140085 | 3286 | 10815 |
| WR089904 | 29156 | 63000 |
| WR090212 | 3539 | 26000 |

As shown in table 3, three out of the eight febrifugine analogues also have inhibition to recombinant plasmodial cyclin-dependent kinase (PfMRK) and mitogen-activated kinase (PfMP-2) indicating possible interference of the parasite cellular signaling pathways.

TABLE 3
Kinase Assays with Analogues.

| Febrifugine/ | | |
|---|---|---|
| Analogues | PFMRK (IC₅₀:μM) | PfMAP-2 (IC₅₀:μM) |
| Febrifugine | — | — |
| WR090212 | — | — |
| WR0B9904 | — | — |
| WR140085 | — | — |
| WR222048 | — | — |
| WWR139672 | — | — |
| WR221232 | ˜113 μM | + ˜? |
| WR059421 | ˜27 μM | + |
| WR088442 | ˜25 μM | + |

(1) Measurement of In Vitro Drug Susceptibility of Different Plasmodium falciparum Populations:

Potential resistance to halofuginone derivatives of P. falciparum isolates were tested. Isolates of P. falciparum stored in liquid nitrogen are thawed and cultivated in RPMI 1640 media with 6% human erythrocytes supplemented with 10% of human serum. The parasite cultures are maintained in an atmosphere of 5% CO₂, 5% O₂ and 90% N₂ at 37° C. for the assay. The semi-automated micro-dilution technique of Desjardins is used to assess the sensitivity of the parasites to febrifugine and halofuginone derivatives. The incorporation of [3H]-hypoxanthine into the parasites is measured as a function of compound concentration to determine EC50 values. Febrifugine, halofuginone and its derivatives, shown above, were tested in the drug susceptibility assay as they are very potent to both P. falciparum chloroquine-sensitive D6-strain and chloroquine-resistant W2-strain with IC₅₀ ranging from 0.4 to 28 ng/ml. The results are shown in FIGS. 2(a) and 2(b) and presented in table 4 below:

TABLE 4
Comparison of in vitro Drug Susceptibility of Malarial
Parasites and Host Cells (IC_{50 ng/ml).}

| | | | Neuronal | Macro |
|---|---|---|---|---|
| Febrifugine | P. falciparum | P. falciparum | Cells | phage |
| Analogs | (W2) | (D6) | NG108 | Cells J774 |
| Febrifugine | 0.53 | 0.34 | 63.50 | 81.00 |
| Halofuginone | 0.15 | 0.12 | 177.06 | 132.25 |
| WR222048 | 0.98 | 0.82 | 878.59 | 498.84 |
| WR139672 | 1.46 | 1.75 | 1157.36 | 392.16 |
| WR059421 | 23.67 | 17.44 | 8933.30 | 492.12 |
| WR221232 | 11.54 | 9.45 | 7996.88 | 612.95 |
| WR140085 | 15.07 | 12.94 | 15479.39 | 425.46 |
| WR090212 | 23.38 | 18.95 | 24820.70 | 2973.31 |
| WR146115 | 28.73 | 21.22 | 67249.23 | 6110.63 |
| WR092103 | 2.93 | 2.38 | 6864.29 | 3605.13 |
| WR089904 | 129.40 | 89.73 | 53251.75 | 1077.73 |
| WR088442 | 245.18 | 192.80 | 35081.19 | 7090.20 |
| WR059424 | 283.68 | 291.29 | 36793.33 | 3012.30 |

(2) Determination of In Vivo Drug Susceptibility of Plasmodium berghei in Mice:

An active animal use protocol mouse models are used to test the above compounds to determine antimalarial activity by using modified Thompson Test. In this test, IRC mice are inoculated intraperitoneally with P. berghei-infected erythrocytes from donor mice that are anesthetized and exsanguinated via cardiac puncture to collect infected blood. The pooled blood is then diluted with normal mouse serum to a concentration of 1×10⁶ P. berghei erythrocytes per inoculum (0.1 ml). The groups of testing and control mice are inoculated with the infected blood on day 0 and then treated with various dosages of halofuginone or the derivatives in aqueous-based vehicles on day 3 through the day required. The drug is administered orally (PO), subcutaneously (SC), intramuscularly (IM), and/or intraperitoneally (IP) up to three times a day, based on the requirements. Blood films and body weights are taken on the third and sixth days post-infection, then at weekly intervals through day 60. Films are Giemsa-stained, examined using light microscopy for the determination of parasitemia. All mice with negative smears at 60 days are considered cured. The data from the in vivo testing verifies the antimalarial efficacy of febrifugine, halofuginone and the halofuginone derivatives in mice and provides new properties of halofuginone and halofuginone derivatives against malarial parasites in vivo.

Test Data 1:

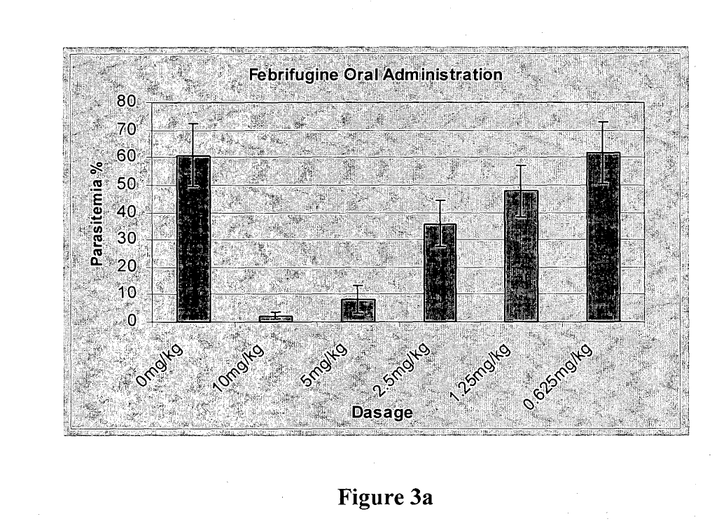

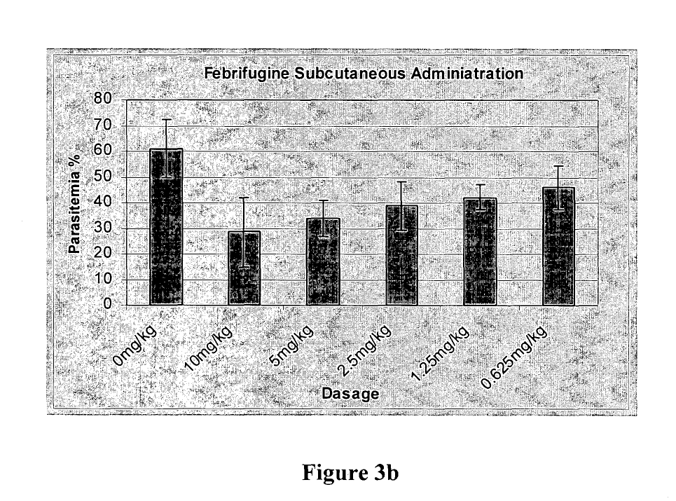

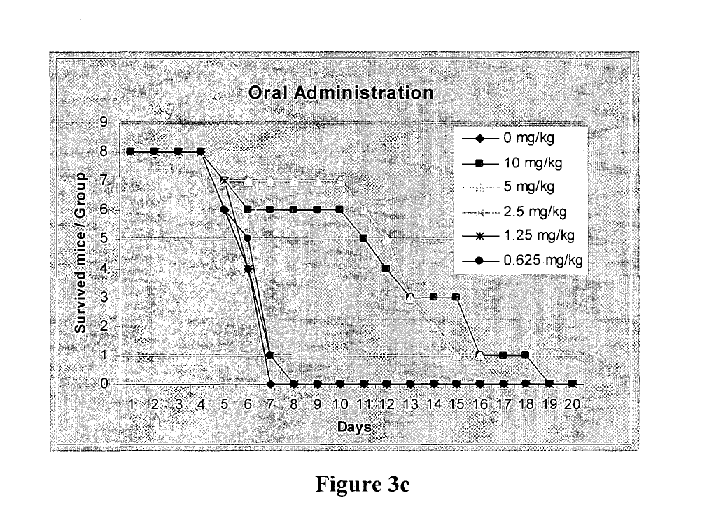

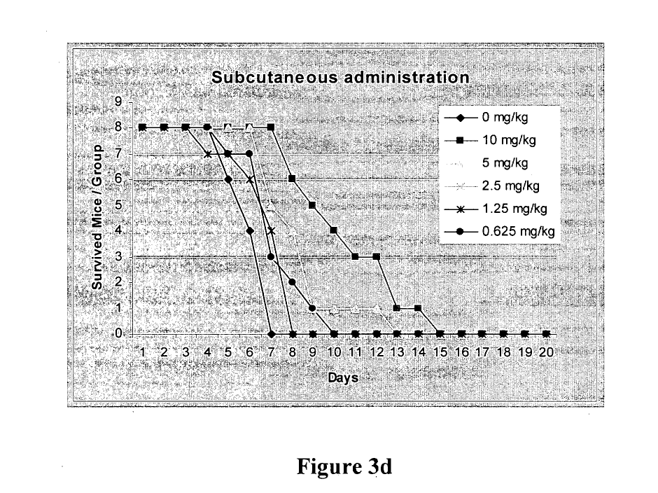

The modified Thompson test was conducted in eleven groups of mice with eight mice per group. The tested mice were inoculated with P. Berghei on day zero and treated with febrifugines on days three with one oral or subcutaneous treatment per day for three days. The oral treatment with febrifugines was found to be more efficacious. The parasitemia of the infected mice was reduced to less than 3% with oral administration of 10 mg/kg febrifugines one/day for three days, while mice with the subcutaneous treatment had 30% of parasitemia at the same does as shown in FIGS. 3(a) through 3(d).

(3) Assessment of Toxic Effects of Halofuginone and the Halofuginone Derivatives:

The side effect of Dichroa febrifuga Lour (Chang Shan) causes nausea and vomiting if overdoes. However, febrifugines at antimalarial dosages do not appear to have such toxic effects in mice.

In order to determine host response to the toxic affects of the drugs at very early stages, one of the best approaches is to measure their cellular and molecular changes to the agonists. Measurements on the DNA damage and cell death of the gastrointestinal tissues and blood cells induced by febrifugine, halofuginone and halofuginone derivatives, using COMET assay (single-cell gel electrophoresis) and TUNEL assay (Terminal Deoxynucleotidyl Transferase Biotin-dUTP Nick labeling) are used. The COMET assay is used to measure the fragmentation of cellular DNA accompanied with severe cell damage and eventual cell death. When the drug-treated cells are embedded in an agarose gel and exposed in an electric field, the fragments of DNA migrate outside of the nucleus region while the intact DNA strands remain inside the nucleus. The distance of the fragment migration is dependent on the extent of DNA damage. The more severe the damage, the longer the distance of fragment migration.

Mouse blood and intraperitoneal cells are collected after the treatment of halofuginone and other derivatives at various dosages and time frames. The blood is mixed with agarose gel and layered on a microscopic slide. The slide is immersed in lysing solution (i.e. 2.5 M NaCl, 100 mM Na₂EDTA, 10 mM Tris-HCl pH 10.1% Na N-lauroyl sarconsinate with 1% Triton X-100 & 10% DMSO) and then in alkaline buffer (300 mM NaOH and 1 mM EDTA, pH 13) to denature the DNA for the detection of single strand DNA damage. After the electrophoresis, the slide is washed with 0.4 M Tris-HCl pH 7.5 three times to neutralize the DNA and dehydrate with methanol followed by ethidium bromide staining. TUNEL assay is done to measure the DNA damage of the gastrointestinal tissues. Here, the DNA breaks are labeled in situ by transfer of biotin-dUTP to free 3′-OH groups of cleaved DNA with modified nucleotides in an enzymatic reaction and detected by fluorescence microscope.

Tests using an assay kit include removing gastrointestinal tissues after drug treatment at different time intervals and dosages and fixed in 4% formaldehyde and embedded in paraffin. Paraffin sections are adhered to poly-L-lysine-treated slides. Deparaffinization and rehydration of the tissue sections are conducted through heating and ethanol/water wash. Slides from the assays are viewed under an Olympus fluorescence microscope and the images captured for digitization analysis. The results provide evidence of the concentrations of febrifugine, halofuginone and halofuginone derivatives that cause toxic effect in the tested mice and subsequently induce tissue cell death and DNA damage.

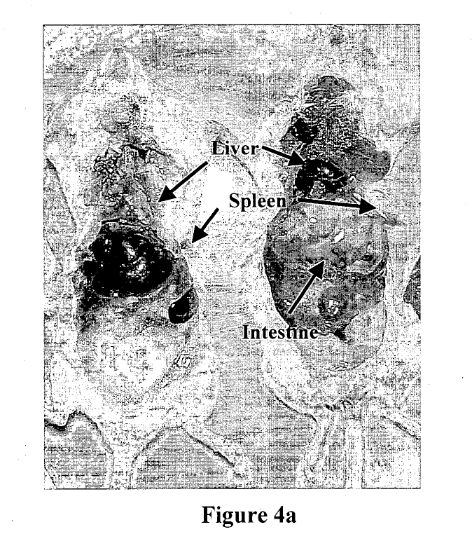

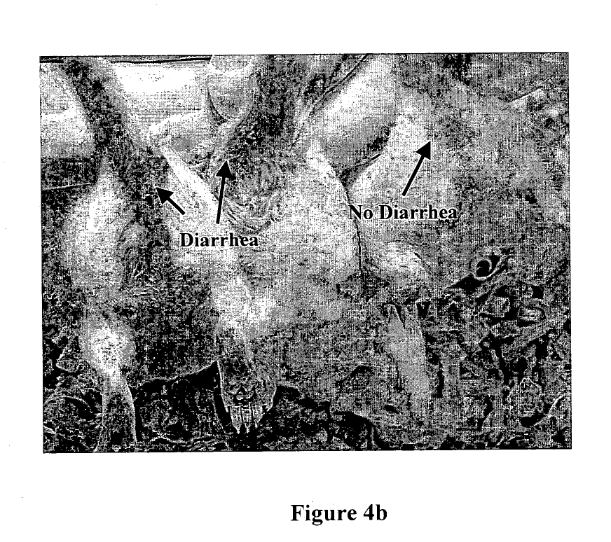

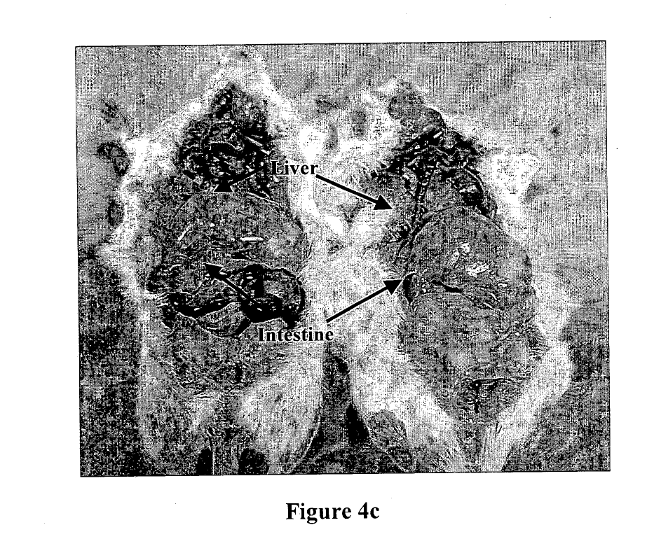

Necropsy analysis as shown in FIG. 4(a) shows the difference between the death caused by malarial parasites and drug toxic effects. The mouse on the left in FIG. 4(a) died of malaria and had dark purple and enlarged liver and spleen indicating the heavy growth of parasites. The mouse on the right in FIG. 4(a) shows that the mouse was killed at the toxic doses of febrifugines showing pale liver and intestine lesions and hemorrhage.

As shown in FIG. 4(b) toxic doses of febrifugines caused diarrhea (left two mice) while the effective treatment doses did not induce diarrhea (right mouse). No vomiting has been observed in the tested mice.

As shown in FIG. 4(c), the comparison between the oral and subcutaneous treatments shows different toxic effects. The oral treatment of toxic doses caused severe gastrointestinal lesions and hemorrhage (left mouse) while the subcutaneous treatment did not induce GI tract injury (right mouse).

(4) Identification of Drug Targets in P. falciparum:

Halofuginone is a known to be a specific collagen gene transcription inhibitor. However, inhibition to other target enzymes by febrifugine, halofuginone and halofuginone derivatives in malarial parasites remain unknown. Quinazoline analogues (the family to which these compounds belong) are known to target dihydrofolate reductase, mammalian EGFR kinase, the stress-activated protein kinase and cyclin dependent kinase. A series of enzymatic assays are used to detect whether the derivatives have inhibition against these enzymes. DHFR assay is conducted using a well-characterized spectrophotometric method. Kinase assays are conducted using radioisotope-labeling technique. The responses of the recombinant kinases to the derivatives are measured in a scintillation b-counter and illustrated using SDS-page gel and PhosphoImager. These tests provide information on the compounds' abilities to interfere with the functions of these enzymes and alter the parasite physiological pathways on a molecular basis.

Test Data 2

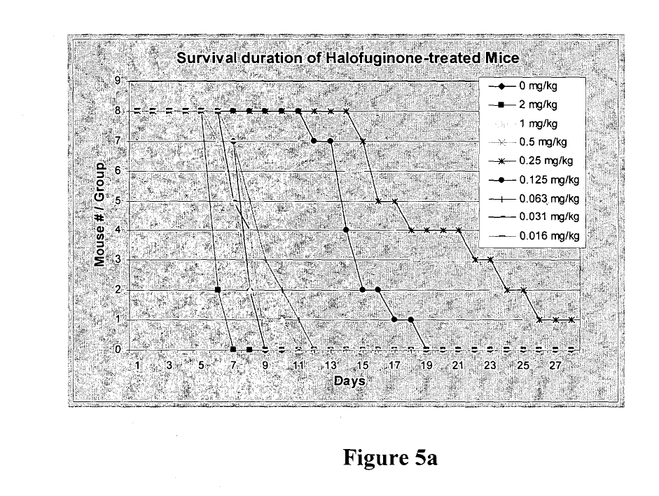

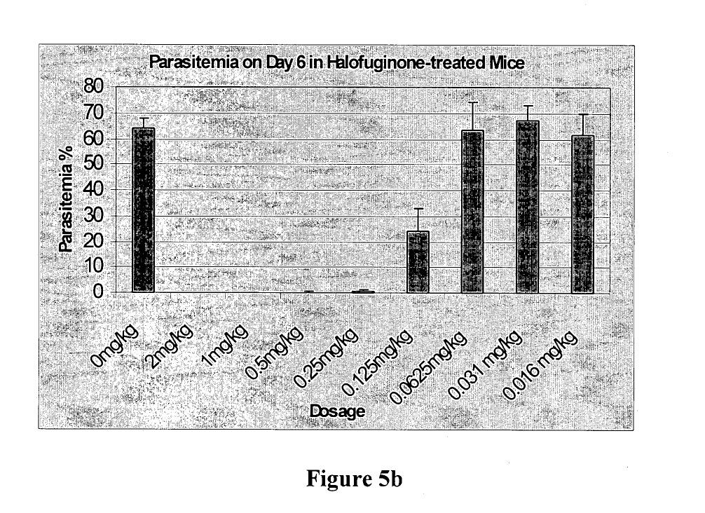

Eighty-eight ICR mice were inoculated with P. berghei, and separated into nine groups. Eight of the infected groups were treated with pH different doses of halofuginone twice a day for eight days. One group was treated with physiological saline as control. As shown in FIG. 5(a), halofuginone extended the mouse survival time at the doses of 0.125, 0.25 and 0.5 mg/kg. Two mice were cured at the doses of 0.25 and 0.5 mg/kg and survived 60 days after the treatment. FIG. 5(b) shows that the parasitemia of the infected mice reduced below 0.5% at the doses of 0.25 and 0.5 mg/kg on day six. The parasites were all cleared at the does of 1 mg/kg on day six. The dose higher than 1 mg/kg killed the tested mice before the parasites.

The experiment of example 2 shows that halofuginone is the most potent analog against malarial parasites in vitro in the group of febrifugine derivatives

(5) Measurement of Drug Disposition in Mouse Models:

Information on the pharmacokinetic properties of febrifugine, halofuginone and halofuginone derivatives is virtually unknown. Exploratory experiments to examine the disposition of febrifugine, halofuginone and its derivatives are conducted using mouse models that are subjected to HPLC with UV detection. The blood samples are collected from ICR mice after administering the drugs at selected dosages and time intervals. The serum is extracted with diethyl ether and the organic phase is evaporated to dryness. The residue is dissolved in the mobile phase, proportionally mixed with a solution of acetonitrile and water, and then separated through a pre-column and analytical column packed with different sizes of dry stationary phase. Standard kinetic models and methods are used to evaluate the data and generate drug concentration-time curves. The results obtained from this experiment not only provide essential information on the basic pharmacokinetic functions of febrifugine, halofuginone and its derivatives in host animals, but also provide necessary information to design drug tests in monkey models.

(6) Detection of In Vivo and In Vitro Immune Responses:

Manny antimalarial drugs have immunosuppressive properties. Therefore it is not recommended for simultaneous vaccination. It is of interest that febrifugines have immunostimulatory activities. The drug increases the production of nitric oxide in the P. berghei-infected mice. The immune modulating activities of halofuginone and its derivatives have never been elucidated. The effects of the compounds on immune responses both in vivo and in vitro are measured. The measurements of the ability of the compounds to induce the production of TNF-a and nitric oxide from immune cells are carried out using a standard microplate assay method. The mouse macrophages are harvested three days after TG-elicitation and cultured in RPMI 1640 supplemented with 10% FBS. The culture medium is removed after drug treatment and mixed with an equal volume of Griess reagent (1% sulfanilamide/0.1% N-(napthyl)-ethylenediamine dihydrochloride/2.5% H₃PO₄). The mixture is incubated at room temperature for ten minutes and then subjected to a microplate reader (absorbance at 510 nm) to determine nitrite concentration. Standard ELISA is used to measure TNF-a secretion of the cultured macrophages. The 96-well microplate coated with the antibodies against murine TNF-a are loaded with the macrophage medium and incubated for an hour. The plate is then exposed to rabbit anti-TNF-a, goat anti-TNF-a, rabbit IgG conjugated with phosphatase and p-nitrophenyl phosphate sequentially followed by absorbance readings at 410 nm. The data obtained from different dosages and time intervals are compared to determine the effect of halofuginone and its analogues to host immune systems.

The present invention therefore shows that halofuginone and other febrifugine derivatives are effective antimalarial agents with halofuginone being the most potent. These results are due to febrifugine and its analogues having lower toxicity to mammalian neuronal and macrophage cells compared to the parasites. The present invention also shows that mammalian neuronal cells are less susceptible to the febrifugine analogues. Additionally, the oral administration of febrifugine in mouse models has better efficacy against malarial parasites than subcutaneous, but produces more irritation to the gastrointestinal tract.

What is claimed is:

1. A method for the treatment of protozoan infections comprising administering a quinazolinone compound in a pharmaceutically effective amount.

2. A method for the treatment of protozoan infections as recited in claim 1, wherein said compound is febrifugine/isofebrifugine extracted from Dichroa febrifuga Lour plant.

3. A method for the treatment of a protozoan infection as recited in claim 2, wherein said administration is selected from the group consisting of oral, subcutaneous, intramuscular, and intraperitoneal.

4. A method for the treatment of protozoan infections as recited in claim 3, wherein said protozoa is of the genus Plasmodium.

5. A method for the treatment of protozoan infections as recited in claim 4, wherein said protozoa is Plasmodium falciparum.

6. A method for the treatment of protozoan infections as recited in claim 4, wherein said protozoa is Plasmodium berghei.

7. A method for the treatment of protozoan infections comprising administering a quinazolinone compound in a pharmaceutical effective amount, said compound having the formula:

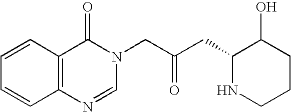

8. A method for the treatment of protozoan infections as recited in claim 7, wherein said administration is selected from the group consisting of oral, subcutaneous, intramuscular, and intraperitoneal.

9. A method for the treatment of protozoan infections as recited in claim 8, wherein said protozoa is of the genus Plasmodium.

10. A method for the treatment of protozoan infections as recited in claim 9, wherein said protozoa is Plasmodium falciparum.

11. A method for the treatment of protozoan infections as recited in claim 9, wherein said protozoa is Plasmodium berghei.

12. A method for the treatment of protozoan infections comprising a quinazolinone compound in a pharmaceutical effective amount, said compound having the formula:

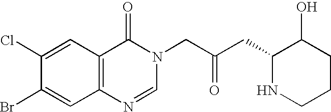

13. A method for the treatment of protozoan infections as recited in claim 12, wherein said administration is selected from the group consisting of oral, subcutaneous, intramuscular, and intraperitoneal.

14. A method for the treatment of protozoan infections as recited in claim 13, wherein said protozoa is of the genus Plasmodium.

15. A method for the treatment of protozoan infections as recited in claim 14, wherein said protozoa is Plasmodium falciparum.

16. A method for the treatment of protozoan infections as recited in claim 14, wherein said protozoa is Plasmodium berghei.

17. A method for the treatment of protozoan infections comprising a quinazolinone compound in a pharmaceutical effective amount, said compound having the formula:

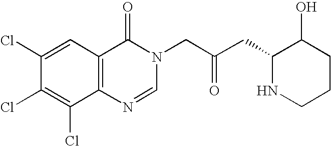

18. A method for the treatment of protozoan infections as recited in claim 17, wherein said administration is selected from the group consisting of oral, subcutaneous, intramuscular, and intraperitoneal.

19. A method for the treatment of protozoan infections as recited in claim 18, wherein said protozoa is of the genus Plasmodium.

20. A method for the treatment of protozoan infections as recited in claim 19, wherein said protozoa is Plasmodium falciparum.

21. A method for the treatment of protozoan infections as recited in claim 19, wherein said protozoa is Plasmodium berghei.

22. A method for the treatment of protozoan infections comprising a quinazolinone compound in a pharmaceutical effective amount, said compound having the formula:

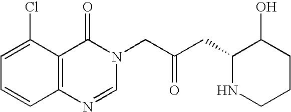

23. A method for the treatment of protozoan infections as recited in claim 22, wherein said administration is selected from the group consisting of oral, subcutaneous, intramuscular, and intraperitoneal.

24. A method for the treatment of protozoan infections as recited in claim 23, wherein said protozoa is of the genus Plasmodium.

25. A method for the treatment of protozoan infections as recited in claim 24, wherein said protozoa is Plasmodium falciparum.

26. A method for the treatment of protozoan infections as recited in claim 24, wherein said protozoa is Plasmodium berghei.

27. A method for the treatment of protozoan infections comprising a quinazolinone compound in a pharmaceutical effective amount, said compound having the formula:

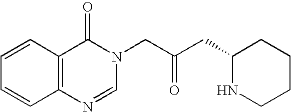

28. A method for the treatment of protozoan infections as recited in claim 27, wherein said administration is selected from the group consisting of oral, subcutaneous, intramuscular, and intraperitoneal.

29. A method for the treatment of protozoan infections as recited in claim 28, wherein said protozoa is of the genus Plasmodium.

30. A method for the treatment of protozoan infections as recited in claim 29, wherein said protozoa is Plasmodium falciparum.

31. A method for the treatment of protozoan infections as recited in claim 29, wherein said protozoa is Plasmodium berghei.

32. A method for the treatment of protozoan infections comprising a quinazolinone compound in a pharmaceutical effective amount, said compound having the formula:

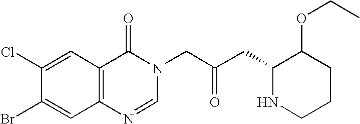

33. A method for the treatment of protozoan infections as recited in claim 32, wherein said administration is selected from the group consisting of oral, subcutaneous, intramuscular, and intraperitoneal.

34. A method for the treatment of protozoan infections as recited in claim 33, wherein said protozoa is of the genus Plasmodium.

35. A method for the treatment of protozoan infections as recited in claim 34, wherein said protozoa is Plasmodium falciparum.

36. A method for the treatment of protozoan infections as recited in claim 34, wherein said protozoa is Plasmodium berghei.

37. A method for the treatment of protozoan infections comprising a quinazolinone compound in a pharmaceutical effective amount, said compound having the formula:

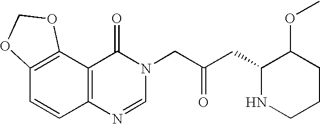

38. A method for the treatment of protozoan infections as recited in claim 37, wherein said administration is selected from the group consisting of oral, subcutaneous, intramuscular, and intraperitoneal.

39. A method for the treatment of protozoan infections as recited in claim 38, wherein said protozoa is of the genus Plasmodium.

40. A method for the treatment of protozoan infections as recited in claim 39, wherein said protozoa is Plasmodium falciparum.

41. A method for the treatment of protozoan infections as recited in claim 39, wherein said protozoa is Plasmodium berghei.

42. A method for the treatment of protozoan infections comprising a quinazolinone compound in a pharmaceutical effective amount, said compound having the formula:

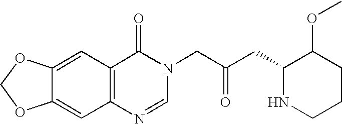

43. A method for the treatment of protozoan infections as recited in claim 42, wherein said administration is selected from the group consisting of oral, subcutaneous, intramuscular, and intraperitoneal.

44. A method for the treatment of protozoan infections as recited in claim 43, wherein said protozoa is of the genus Plasmodium.

45. A method for the treatment of protozoan infections as recited in claim 44, wherein said protozoa is Plasmodium falciparum.

46. A method for the treatment of protozoan infections as recited in claim 44, wherein said protozoa is Plasmodium berghei.

47. A method for the treatment of protozoan infections comprising a quinazolinone compound in a pharmaceutical effective amount, said compound having the formula:

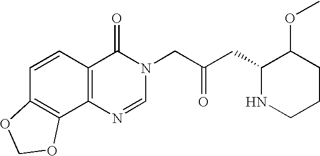

48. A method for the treatment of protozoan infections as recited in claim 47, wherein said administration is selected from the group consisting of oral, subcutaneous, intramuscular, and intraperitoneal.

49. A method for the treatment of protozoan infections as recited in claim 48, wherein said protozoa is of the genus Plasmodium.

50. A method for the treatment of protozoan infections as recited in claim 49, wherein said protozoa is Plasmodium falciparum.

51. A method for the treatment of protozoan infections as recited in claim 50, wherein said protozoa is Plasmodium berghei.

52. A method for the treatment of protozoan infections comprising a quinazolinone compound in a pharmaceutical effective amount, said compound having the formula:

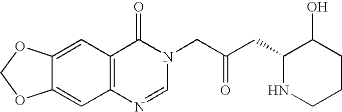

53. A method for the treatment of protozoan infections as recited in claim 52, wherein said administration is selected from the group consisting of oral, subcutaneous, intramuscular, and intraperitoneal.

54. A method for the treatment of protozoan infections as recited in claim 53, wherein said protozoa is of the genus Plasmodium.

55. A method for the treatment of protozoan infections as recited in claim 54, wherein said protozoa is Plasmodium falciparum.

56. A method for the treatment of protozoan infections as recited in claim 54, wherein said protozoa is Plasmodium berghei.

57. A method for the treatment of protozoan infections comprising a quinazolinone compound in a pharmaceutical effective amount, said compound having the formula:

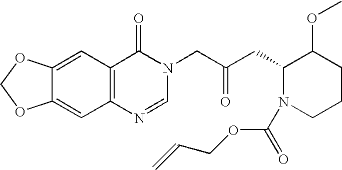

58. A method for the treatment of protozoan infections as recited in claim 57, wherein said administration is selected from the group consisting of oral, subcutaneous, intramuscular, and intraperitoneal.

59. A method for the treatment of protozoan infections as recited in claim 58, wherein said protozoa is of the genus Plasmodium.

60. A method for the treatment of protozoan infections as recited in claim 59, wherein said protozoa is Plasmodium falciparum.

61. A method for the treatment of protozoan infections as recited in claim 59, wherein said protozoa is Plasmodium berghei.

62. A method for the treatment of protozoan infections comprising a quinazolinone compound in a pharmaceutical effective amount, said compound having the formula:

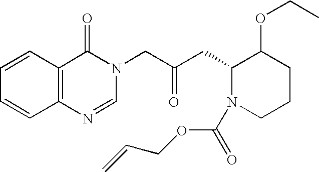

63. A method for the treatment of protozoan infections as recited in claim 62, wherein said administration is selected from the group consisting of oral, subcutaneous, intramuscular, and intraperitoneal.

64. A method for the treatment of protozoan infections as recited in claim 63, wherein said protozoa is of the genus Plasmodium.

65. A method for the treatment of protozoan infections as recited in claim 64, wherein said protozoa is Plasmodium falciparum.

66. A method for the treatment of protozoan infections as recited in claim 64, wherein said protozoa is Plasmodium berghei.

67. A method for the treatment of protozoan infections comprising a quinazolinone compound in a pharmaceutical effective amount, said compound having the formula:

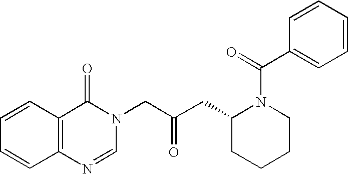

68. A method for the treatment of protozoan infections as recited in claim 67, wherein said administration is selected from the group consisting of oral, subcutaneous, intramuscular, and intraperitoneal.

69. A method for the treatment of protozoan infections as recited in claim 68, wherein said protozoa is of the genus Plasmodium.

70. A method for the treatment of protozoan infections as recited in claim 69, wherein said protozoa is Plasmodium falciparum.

71. A method for the treatment of protozoan infections as recited in claim 69, wherein said protozoa is Plasmodium berghei.
